(19) United States
(12) Patent Application Publication
(10) Pub. No.: US 2008/0215054 A1
(43) Pub. Date: Sep. 4, 2008

(54) Devices and Methods for Minimizing the Hemorrhage from and Minimizing Infection of a Divided Sternum During Cardiac Surgery

(75) Inventor: John T.M. Wright, Denver, CO (US)

Correspondence Address: SWANSON & BRATSCHUN, L.L.C., 8210 SOUTHPARK TERRACE, LITTLETON, CO, 80120, US

(73) Assignee: GENESEE BIOMEDICAL, INC., Denver, CO (US)

(21) Appl. No.: 12/020,403

(22) Filed: Jan. 25, 2008

Related U.S. Application Data
(60) Provisional application No. 60/886,887, filed on Jan. 26, 2007.

(51) Int. Cl. A61B 17/58; A61M 29/00

(52) U.S. Cl. 606/60; 606/191

(57) ABSTRACT

A device for capping a severed sternum. The device comprises an end wall configured to extend along a length of severed sternum. A gasket is attached to the end wall configured to abut at least part of the length of a severed sternum when deployed thereon.

### RELATED APPLICATIONS

This application claims priority from U.S. Provisional Patent Application Ser. No. 60/886,887, filed Jan. 26, 2007, entitled “Devices and Methods for Minimizing the Hemorrhage from and Minimizing Infection of a Divided Sternum During Cardiac Surgery,” which is hereby incorporated by reference.

### TECHNICAL FIELD

The present disclosure relates generally to methods and devices associated with cardiac and surgery and, more particularly, to methods and devices for minimizing the loss of blood from and minimizing risk of infection of a severed sternum during cardiac surgical procedures.

### BACKGROUND

A full median sternotomy is the most common procedure currently performed for providing surgical access to the heart and coronary arteries. A sternotomy, however, is highly invasive. The patient's skin is incised at the midline overlying the chest and the patient's sternum is cut, usually using a pneumatically or electrically powered surgical saw, typically along its entire length. In cardiac valve surgery the cut edges of the sternum are then spread with metal retractors, exposing a large cavity to allow surgery to be performed on the heart. Representive retractors are described in U.S. Pat. Nos. 5,772,583 and 6,206,828. Generally, such retractors use two substantially parallel retractor blades that remain generally at the same height in the operative position. However, in coronary bypass surgery, following the sternotomy, the left portion of the severed sternum is lifted and laterally retracted to gain access to the left internal mammary artery (IMA), which is commonly the principle bypass graft of choice. Several types of retractors are available for IMA exposure. Commonly two “rake” shaped retraction members are used such as are described in U.S. Pat. No. 6,689,053. Some surgeons use the right IMA in addition to the left IMA.

Sternotomy typically results in hemorrhage from the cut sternal edges where the bone marrow is exposed. This loss of blood is unacceptable for at least two reasons. First it may obstruct or obliterate the view of the surgical team when performing the surgical procedure such as taking down the Internal Mammary Artery (IMA) during coronary artery bypass surgery. Second, the lost blood must be replaced, either by transfusion or by returning it to the cardiotomy reservoir of the heart-lung machine via a sucker (that unfortunately traumatizes the blood). Thus, hemostatis is required before the operation may proceed. Even more important is that hemorrhage has to be arrested before a systemic anticoagulant (e.g. heparin) is administered just prior to the onset of cardiopulmonary bypass.

To minimize hemorrhage from the severed sternum, sternal waxes, powders and the like have been developed to be applied to the bleeding surfaces of the sternum halves following the splitting of the sternum, usually in association with the use of electo-cautery to quell the larger bleeding sites. These substances and techniques help to inhibit and/or otherwise reduce the hemorrhage, but may contribute to the incidence of mediastinal infection and may inhibit healing and bone reunification.

It would thus be beneficial if these substances and electro-cautery could be eliminated from the sternum halves during the surgical procedure. However, the current state of the art is lacking in this regard. Currently these substances are left in the sternum (i.e., between the sternal halves) following the surgical procedure. The residues can cause contamination of the blood cells that may lead to additional post operative procedures and treatments. Also, these substances have proven to be less than effective in performing their intended function, i.e., controlling hemorrhage.

In an attempt to address the problem LiDonnici, U.S. Pat. No. 7,011,628, describes a device intended to stanch the effusion of blood from the exposed ends of the sternal halves of an incised sternum. The LiDonnici devices comprises a substantially U-shaped cross-section cap intended to cap or cover the severed sternum halves. When the sternum is slit using a free-hand reciprocating saw with a narrow blade (much like jigsaw) the cut edge tends to be wavy rather than absolutely straight, and the cut may not be at right angles to the anterior surface of the sternum. The LiDonnici device is ineffective at forming a tight seal between a sternal device and the undulating cut edges of the sternum.

Sternotomy occasionally results in a post operative mediastinal infection. The incidence of mediastinal infection is 0.4-5%. Mediastinal infection is a feared complication of cardiac operations as patients with mediastinal infections face a protracted hospital stay at best and have a mortality rate as high as 20%-40% (Marggraf, et al. (2003) European Journal of Surgery 16, S9:12-16). It is believed that some sternal infections are caused by contamination (airborne or otherwise) of the exposed bone marrow.

Accordingly, a continuing need exists for improved methods and devices for minimizing the hemorrhage from a severed sternum during cardiac surgical procedures and further protecting the exposed edge of the sternum to prevent mediastinal infection.

### SUMMARY

One aspect is a device for capping a severed sternum. The device comprises an end wall configured to extend along a length of severed sternum. A gasket is attached to the end wall configured to abut at least part of the length of a severed sternum when deployed thereon. In one embodiment the device further comprises anchoring means for attaching the end wall to the sternum with the gasket engaging at least a part of the length of the sternum. The anchoring means preferably provides sufficient pressure on the severed sternum to stanch blood flow. The anchoring means may comprise at least one of a nylon cable tie or stainless steel wire. Embodiments of the device may comprise means for stiffening the end wall. The device may further comprise at least one of a top and a bottom wall extending lengthwise of the end wall adjacent to the gasket.

Another aspect is a method of capping the exposed medial sides of a sternal half of a longitudinally divided sternum. The method comprises providing a pair of elongate end walls having a gasket attached to a surface thereof and placing the gaskets under lateral pressure against each exposed sternal half. In one embodiment the method further comprises harvesting an internal mammary artery (IMA) from a patient's chest. The method may further comprise providing a sternal retractor, applying blades of the sternal retractor to the elongate end wall opposite the gasket and applying pressure to the blades to separate the sternum halves. In one embodiment the method further comprises securing each elongate end wall under a blood stanching pressure to each sternal half. The securing step may be performed using at least one of a nylon cable tie or a stainless steel wire.

The device and method disclosed and claimed herein provides a secure and reliable means for stanching blood flow from an incised sternum and for minimizing the risk of mediastinal infection to a patient. The device can be easily and inexpensively manufactured, thus making the many advantages available at minimal cost.

### BRIEF DESCRIPTION OF THE DRAWINGS

FIG. 1 is a rear perspective view of a device, according to an embodiment of the present disclosure, for covering an exposed end of a sternal half of a longitudinally divided sternum;

FIG. 2 is a front perspective view of a device of FIG. 1;

FIG. 3 is a front elevation view the device of FIG. 1.

FIG. 4 is a side elevation view of the device of FIG. 1

FIG. 5 is a cross sectional view taken along line AA of FIG. 3.

FIG. 6 shows a cross sectional view the device and the sternum with the device tightly attached to the sternum with stainless wire to form a blood-tight seal between the sternum by means of a stainless steel surgical suture.

FIG. 7 shows a cross sectional view the device and the sternum with the device tightly attached to the sternum with a cable tie to form a blood-tight seal between the sternum by means of a cable tie.

FIG. 8 shows a isometric view the device and the sternum with the device tightly attached to the sternum with stainless steel wire to form a blood-tight seal between the sternum by means of a stainless steel surgical suture.

FIG. 9 shows a isometric view the device and the sternum with the device tightly attached to the sternum with a cable tie to form a blood-tight seal between the sternum by means of a tensioned cable tie.

### DETAILED DESCRIPTION

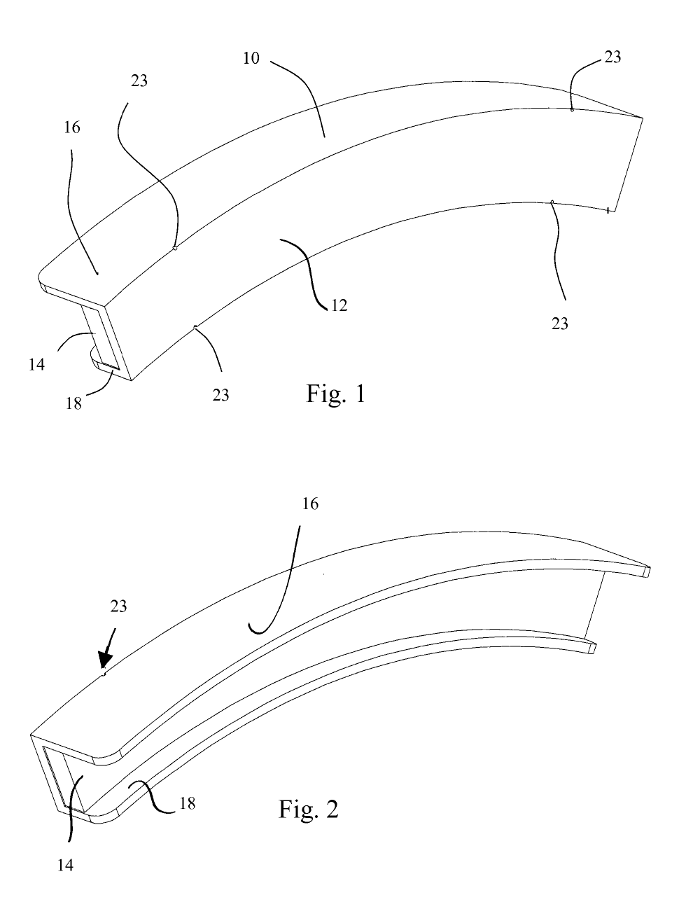

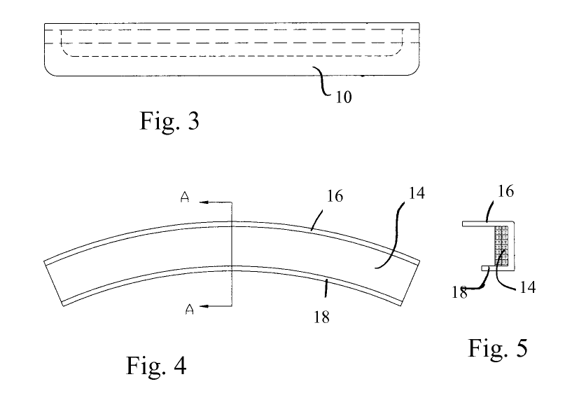

A device 10 for minimizing hemorrhage from an exposed sternal half of a sternum formed during a sternotomy is illustrated in FIG. 1. The device includes an elongate end wall 12 having a gasket 14. As illustrated, the end wall may be curved as desired to fit a sternum half. The end wall and gasket have a size and a dimension to cover or least partially cover an exposed end of a sternal half, wherein suitable pressure is applied between the gasket of the device and the cut edge of the sternum the of the device to form a hydrostatic seal that the stanches effusion of blood from the exposed end of the sternal half, and obstructs the ingress of bio-contaminants.

The device may further include an upper wall 16 which may be integrally formed with and extend orthogonally from an upper edge of the end wall 12; and a lower wall 18 which may also be integrally formed with and extend orthogonally from a lower edge of the end wall. The upper wall and the lower wall may extend along the first and second ends of the end wall. The upper wall and the lower wall serve to stiffen the device and minimize distortion when pressure is applied. Alternatively, the upper and lower walls maybe be omitted and only the end wall 12 and gasket 14 comprise the device. Such an embodiment may include stiffeners such as rearwardly projecting supports or the end wall may be made of rigid material such as stainless steel of gauge sufficient to prevent bending. Alternatively, guides or posts may extend from the end wall in place of and in the same direction of the continuous walls 16, 18.

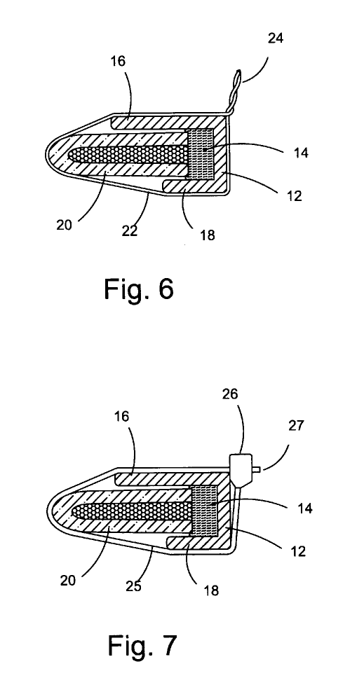

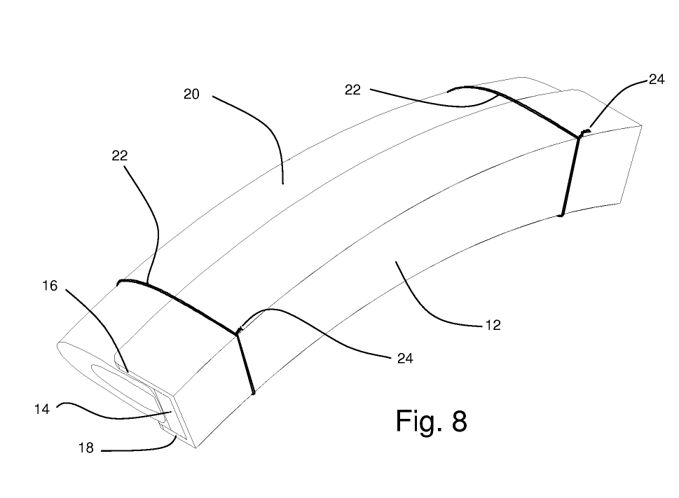

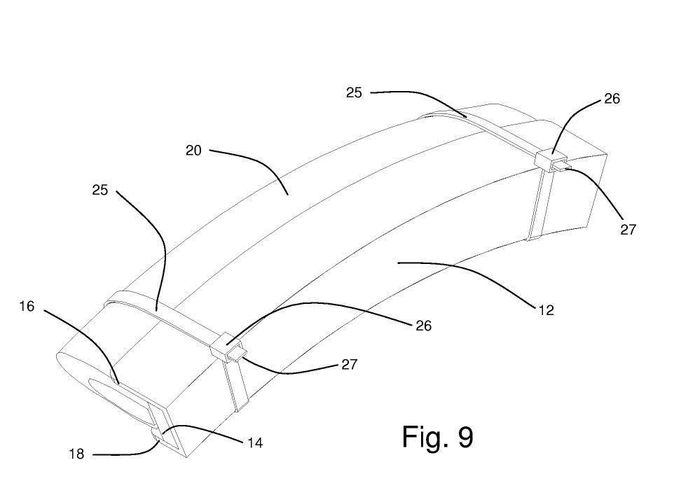

The device may include anchoring means proximal to the axial ends of each device extending laterally from the end wall and wrapping around the lateral portions of a sternum, or intra-costal spaces, with the device engaging the sternum. For example, the anchoring structure may be a stainless steel wire 20, similar to that generally used to re-approximate and firmly hold the sternum 22 at the conclusion of the surgery. Size 5 wire (0.032″ diameter) is preferred by many surgeons. The wire maintains pressure between the cut edge of the sternum 22 and the gasket thus providing a substantially hemostatic seal. The anchoring structures 20 may be removably connected to the end wall as illustrated, in FIGS. 6-9, by engaging notches 23 near the ends of the device (See FIG. 1). As in a sternal closure, the opposing ends of the anchoring sutures are pulled tightly together and the free ends twisted together at 24 as shown in FIG. 6. Sufficient pressure is applied to stanch blood flow. Alternatively, a suitable medical grade Nylon or other suitable material cable tie 25 (for example, see U.S. Pat. No. 3,368,247) may be used to maintain sufficient force between the device and the cut edge of the sternum to form a haemostatic seal, as illustrated in FIGS. 7 and 9. The thin tongue of the cable tie is passed through the body 26, tensioned and excess tongue cut off to leave a small protruding end 27. In either case the restraining members (wires or cable ties) will be cut and removed with the sternal protection device immediately prior to sternal closure.

The end wall may be fabricated from at least one of a biocompatible plastic, hard elastomer or metal such as stainless steel, aluminum, titanium, or other suitable metal. The gasket should be made of a resilient deformable material suitable for minimizing the flow of fluid from the severed sternum. For example, the flexible gasket may be of a biocompatible polyethylene closed cell foam of about 3 mm-6 mm or more in thickness. Other suitable gasket materials include a soft biocompatible low durometer silicon elastomer or synthetic rubber.

The device may have a “U-shaped” transverse cross-sectional profile, wherein the gasket surface contacting the exposed end of the sternal half is substantially flat. The shape and dimensions of the end wall desirably are determined by studying sagittal plane computer tomographic (CT) images for male and female patients of various sizes. See Maddern, et al. (1993) Radiology 186:665-670. Alternatively magnetic resonance imaging could be used to determine device sizes and shapes. See Aslam, et al. (2002) British Journal of Radiology 75:627-634. In one embodiment, several adult and pediatric sized devices would be provided, so that the surgeon could use a device appropriate to the patient. The upper and lower walls may have a thickness of about 2.5 mm. The end wall may have a thickness of about 3 mm, and the flexible gasket may have a thickness of about 6 mm. The device may be fabricated from the same materials discussed above.

Desirably, the distance between the upper and lower walls of the device is about 20 mm. In an alternative embodiment the maximum distance between the upper and lower walls of the device intended to be placed at the upper end of the sternum (the manubrium) is about 20 mm with a distance of about 12 mm at the lower end (the xyfoid).

In use, a pair of devices having flexible gaskets for minimizing hemorrhage from the exposed medial sides of the sternal halves are placed with the gaskets abutting the sternal halves. The device is placed under lateral pressure against each exposed end of each sternal half sufficient to stanch blood flow. Following the harvesting of an IMA vessel(s), blades of the sternal retractor apply pressure to the devices.

The method of use may further include the step of imaging or estimating the size of the sternum to determine the size of the device required for the surgical procedure. The method may further include the steps of placing the blades of a surgical retractor, when in an approximated position, between the devices placed over the exposed ends of the sternal halves and manipulating the retractor to separate the blades of the surgical retractor and spread the sternal halves apart.

In a sternotomy wherein the sternum of a patient has been longitudinally incised along at least a portion thereof, thereby exposing and allowing two opposing sternal halves to be separated laterally, the improvement includes the step of providing a pair of caps for minimizing hemorrhage from the exposed sternal halves of the sternum. The improvement further includes placing a cap on each exposed sternal half such that the sternal half is abutted by the flexible gasket of the cap.

Each cap includes an end wall interconnecting the upper and lower walls, the end wall having a flexible gasket. An upper wall and a lower wall may combine with end wall to bound a space. In such an embodiment the upper wall and the lower wall define an opening through which the sternal half is receivable into the space of the cap.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. All references cited herein are incorporated in their entirety by reference.

What is claimed is:

1. A device for capping a severed sternum comprising:
an end wall configured to extend along at least part of the length of a severed sternum; and
a gasket attached to the end wall configured to abut the at least part of a length of a severed sternum.

2. The device of claim 1 further comprising anchoring means for attaching the end wall to the sternum with the gasket engaging the at least a part of the length of the severed sternum.

3. The device of claim 2 wherein the anchoring means comprises at least one of a nylon cable tie or stainless steel wire.

4. The device of claim 1 further comprising means for stiffening the end wall.

5. The device of claim 1 further comprising at least one of a top and bottom wall extending lengthwise of the end wall adjacent to the gasket.

6. A method of capping the exposed medial sides of a sternal half of a longitudinally divided sternum comprising:
providing a pair of elongate end walls having a gasket attached to a surface thereof; and
placing the gasket under lateral pressure against each exposed sternal half.

7. The method of claim 6 further comprising harvesting an internal mammary artery (IMA) from a patient's chest.

8. The method of claim 7 further comprising providing a sternal retractor, applying blades of the sternal retractor to the elongate end wall opposite the gasket and applying pressure to the blades to separate the sternal halves.

9. The method of claim 6 further comprising securing each elongate end wall under a blood stanching pressure to each sternal half.

10. The method of claim 9 wherein the securing step is performed using at least one of a nylon cable tie or a stainless steel wire.
